(19) United States
(12) Patent Application Publication
(10) Pub. No.: US 2026/0295108 A1
(43) Pub. Date: Oct. 1, 2026

(54) Disinfecting apparatus

(71) Applicant: Specialist Health Solutions Limited, Peterborough (GB)

(72) Inventor: Tautvydas Karitonas, Peterborough (GB)

(21) Appl. No.: 19/633,076

(22) Filed: Mar. 30, 2026

(30) Foreign Application Priority Data
Mar. 31, 2025 (GB) 2504784.6
Mar. 31, 2025 (GB) 2504786.1
Mar. 31, 2025 (GB) 2504787.9

(51) Int. Cl. A61L 9/14

(52) U.S. Cl. CPC A61L 9/14; A61L 2209/131; A61L 2209/132; A61L 2209/134; A61L 2209/15; A61L 2209/211

(57) ABSTRACT

A nebuliser assembly for a disinfecting apparatus is provided. The, comprising: a nebuliser comprising: an atomisation plate having a front surface and a back surface and a plurality of holes extending therebetween; a transducer configured to vibrate the atomisation plate; a fluid retaining substrate arranged against the back surface of the atomisation plate, such that during vibration of the atomisation plate, fluid retained in the fluid retaining substrate is forced through the plurality of holes and ejected from the front surface of the atomisation plate.

### CROSS-REFERENCE TO RELATED APPLICATION

The present application claims priority to GB 2504784.6, GB 2504786.1, and GB 2504787.9, each filed on Mar. 31, 2025, and titled “Disinfecting Apparatus,” the disclosures of which are incorporated by reference in their entirety.

### FIELD OF THE INVENTION

The present invention relates to an apparatus for disinfecting a space, in particular an apparatus that disperses hydrogen peroxide during a disinfecting cycle.

### BACKGROUND

Hydrogen peroxide is known to be effective as a disinfecting fluid. Therefore, it has been considered to use devices that disperse hydrogen peroxide into a space (e.g., a room or vehicle) in order to disinfect surfaces. Such devices are typically filled manually with hydrogen peroxide (e.g., through an opening into a reservoir). Then, once a disinfecting cycle is initiated, an atomiser connected to the reservoir atomises the fluid so it can disperse throughout the space. For example, once dispersed into the air, the fluid may condense onto surfaces within the space to disinfect them.

However, existing devices have the following problems.

First, handling of hydrogen peroxide requires care. Existing apparatuses that require manual refilling are susceptible to spillages. Furthermore, such refilling steps take more time and expertise to carry out. Therefore, there is a desire to facilitate the refilling of disinfecting apparatuses in a manner that reduces complexity and risk of spillage.

Secondly, once a disinfection cycle is started within a space, it is important for the cycle to fully complete before humans re-enter the space. Existing apparatuses do not typically implement any procedures to ensure that this occurs. This means that it is possible for the devices to only partially complete a disinfection cycle, thereby require time-consuming refilling and resetting operations to occur.

Thirdly, existing apparatuses only allow minimal control over the amount of disinfecting fluid that is ejected from the atomiser. This can result in insufficient disinfection of the space (when not enough is used) and/or waste of disinfecting fluid (e.g., when too much is used).

Fourth, there is a desire for such apparatuses to be kept as small as possible while still enabling larger spaces to be disinfected. Ideally, a disinfection apparatus should be small enough to fit within smaller spaces (e.g., biological safety cabinets) while still offering effective disinfection when used in larger spaces (e.g., rooms or vehicles).

Therefore, it is an object of the present invention to address at least some of the problems discussed above.

### SUMMARY OF INVENTION

According to a first aspect of the present invention, there is provided an apparatus for disinfecting a space, comprising: a primary reservoir configured to contain a volume of disinfecting fluid corresponding to a disinfection cycle, the primary reservoir comprising a connection interface configured to facilitate a fluid connection with a secondary reservoir, thereby enabling replenishment of fluid within the primary reservoir; and a nebuliser assembly fluidly connected to the primary reservoir and configured to atomise the disinfecting fluid according to the disinfection cycle in order to disinfect the space.

When carrying out disinfection processes (e.g., to remove harmful viruses and/or bacteria), it is important that a disinfection cycle runs fully to completion once it has started. If the disinfection cycle is only partially completed, then it is not necessarily safe for an operator to re-enter the space. Therefore, by providing a primary reservoir with a volume that is sufficient to carry out the complete disinfection cycle, it can be ensured that the apparatus is always able to carry out a full cycle, without an operator needing to re-enter the space after partial completion (such as to manually replenish disinfecting fluid). A typical disinfection cycle may require a volume of about 60 mL to about 120 mL. The volume of the primary reservoir may be about 120 mL.

The apparatus may further comprise a sensor configured to detect whether sufficient disinfecting fluid is contained within the primary reservoir. In this way, it can be determined whether a disinfection cycle can be fully completed using the fluid present within the primary reservoir. If there is insufficient disinfecting fluid, then the cycle may be prevented from starting and/or an alert may be provided to refill the primary reservoir (e.g., by replacing the secondary reservoir).

Preferably, the sensor is a binary sensor configured to indicate whether sufficient or insufficient disinfecting fluid is present within the primary reservoir. Advantageously, such a sensor can be relatively simple, while still ensuring that disinfection cycles are completely carried out. This may reduce the cost and time associated with manufacturing and/or repairing the apparatus.

Preferably, the sensor comprises: an optical source, an optical sensor, and a transparent member arranged in the primary reservoir, wherein the transparent member is configured to: allow light from the optical source to pass through the transparent member when the transparent member is submerged in disinfecting fluid; and reflect light from the optical source to the optical sensor by total internal reflection when the transparent member is not submerged in the disinfecting fluid.

Since the difference in refractive index (e.g., between the material of the transparent member and the surrounding interior of the reservoir) decreases when the transparent member is submerged in fluid, the critical angle of the optical system changes. In this way, the transparent member only directs light from the optical source back to the optical sensor when it is not submerged. Therefore, based on the measurement by the optical sensor, it is possible to determine whether the primary reservoir contains sufficient fluid. The transparent member may be made of glass. Alternatively, the transparent member may be made from another transparent material. This sensor may be referred to herein as a “total internal reflection sensor”. Preferably, the transparent member provides at least a portion of the external surface of the primary reservoir. The optical source and optical sensor may be positioned on the apparatus at a position that aligns with the transparent member. In this way, no electrical components are required as part of the primary reservoir, which decreases the manufacturing costs. Alternatively, all parts of the sensor may be provided as part of the primary reservoir.

The apparatus may comprise control electronics, wherein the control electronics are configured to prevent initiation of the disinfection cycle when the sensor indicates that insufficient disinfecting fluid is contained within the primary reservoir. In this way, the apparatus is able to prevent a situation where a disinfection cycle is only half completed.

Preferably, the connection interface is provided on an upper surface of the primary reservoir, thereby enabling the secondary reservoir to replenish the primary reservoir by gravity, during use. Advantageously, no additional pumps are required in order to fill the primary reservoir using the secondary reservoir.

Preferably, the connection interface comprises a first connector and a second connector, each configured to interface with a corresponding port of the secondary reservoir. Advantageously, by providing two connectors and ports, disinfecting fluid and air can flow separately to each other in opposite directions.

Preferably, the first connector and/or the second connector is provided by a needle connector. In this way, when the secondary reservoir is attached to the primary reservoir, the needles can extend into corresponding ports of the secondary reservoir, preferably piercing a membrane. The membrane is preferably a resealable membrane. The term “resealable” preferably indicates that the secondary reservoir can be removed from the apparatus while subsequently retaining a seal between the interior of the reservoir and the surroundings. Alternatively, the membrane may be a single use membrane.

The first needle connector may have a diameter from about 5.5 mm to about 10 mm. The first needle connector may enable flow of disinfecting fluid from the secondary reservoir to the primary reservoir. The first needle connector may be referred to as the “fluid needle”. Advantageously, by using a diameter (e.g., internal diameter) of at least 5.5 mm, there is a high certainty of fluid flow through the needle. By having a diameter less than 10 mm, airflow in an opposite direction to the fluid flow is inhibited due to surface tension. In this way, the air flows substantially only through the second needle connector. The second needle connector may be referred to as the “air needle”. Preferably, the second needle connector has a diameter that matches a diameter of a corresponding membrane of the secondary reservoir. The (e.g., internal) diameter of the second needle connector may be about 3 mm.

In an alternative approach, a single needle connector may be used to transmit disinfecting fluid and air in opposite directions. Preferably, such a needle connector has a diameter (e.g., internal diameter) of at least 10 mm.

The apparatus may further comprise a secondary reservoir having a respective connection interface configured to connect to the connection interface of the primary reservoir.

The secondary reservoir may be a single use reservoir. For example, the single use reservoir may not have a refilling port. Alternatively, the secondary reservoir can be refillable.

Preferably, the connection interface of the secondary reservoir comprises a first port configured to supply fluid to the primary reservoir, and a second port configured to receive displaced air from the primary reservoir.

Preferably, the first port and/or the second port comprise a membrane pierceable by respective a needle connector of the connection interface when the secondary reservoir is connected to the primary reservoir. In this way, the secondary reservoir can be kept sealed up until the point of use, and only unsealed during the connection process at the connection interface. The membrane may comprise PTFE and/or silicon.

Preferably, the secondary reservoir comprises a valve assembly configured to enable airflow into and/or out of the secondary reservoir. During use of the apparatus, disinfecting fluid is consumed, which decreases the volume of fluid contained in the primary reservoir and/or secondary reservoir. To replace this fluid, it is advantageous to allow air to flow into the primary reservoir and/or secondary reservoir through the valve assembly. Furthermore, due to changes in atmospheric conditions (e.g., ambient temperature), it is beneficial to allow gradual pressure equalisation with the surrounding air.

Preferably, the valve assembly comprises a first valve movable between a first position where airflow is inhibited, and a second position where airflow is enabled. The first valve may be moved by an operator prior to initiation of a disinfection cycle. The first valve may be moved between its first and second positions by twisting.

Preferably, the valve assembly comprises a second valve having a membrane, wherein the membrane is gas permeable and liquid impermeable. Advantageously, the second valve enables gradual pressure equalisation in apparatus such as due to changes in atmospheric conditions (e.g., due to changes in atmospheric temperature or pressure). The membrane of the valve assembly may be a hydrophobic material. The membrane of the valve assembly may comprise a Gore-Tex® material. Advantageously, it is possible to enable air to flow into and/or out of the apparatus while preventing leakage of disinfecting fluid. The second valve allows this to occur even when the first valve is in its first position.

Preferably, the valve assembly is connected to the second port by an air channel. This may allow refilling of the primary reservoir with fluid from the secondary reservoir to be controlled by operation of the valve assembly. Alternatively, air can bubble from the second port to the top of the secondary reservoir to reach the valve assembly.

Preferably, the apparatus is configured to estimate a volume of fluid within the secondary reservoir based on usage data. In this way, an operator may be provided with a warning when the secondary reservoir needs to be replaced. For example, a new secondary reservoir may have a volume of about 270 ml in the secondary reservoir. During use, the apparatus (e.g., a controller) may measure the rate of consumption rate (e.g., using a flow sensor or other suitable type of sensor) and substrate the estimated volume of disinfecting fluid from the initial volume.

Preferably, the secondary reservoir comprises a writable RFID tag, and the apparatus is configured to update the RFID tag with the estimate of the volume of fluid. In this way, the estimated remaining amount of disinfecting fluid is stored by the secondary reservoir. In this way, if a (e.g., reusable) secondary reservoir is removed from the apparatus and returned later, the apparatus can determine the amount of fluid remaining and continue to maintain an accurate estimate.

According to a second aspect of the present invention, there is provided a nebuliser assembly for a disinfecting apparatus, comprising: a nebuliser comprising: an atomisation plate having a front surface and a back surface and a plurality of holes extending therebetween; a transducer configured to vibrate the atomisation plate; a fluid retaining substrate arranged against the back surface of the atomisation plate, such that during vibration of the atomisation plate, fluid retained in the fluid retaining substrate is forced through the plurality of holes and ejected from the front surface of the atomisation plate.

Advantageously, by providing a fluid retaining substrate against the back surface of the atomisation plate, the amount of disinfecting fluid that is ejected from the nebuliser can be precisely controlled (e.g., by adjusting the level of saturation of the fluid in the substate). By contrast, where fluid is contained in a reservoir that directly contacts atomisation plate, then there is very little control over the amount of fluid that is ejected.

During operation of the nebuliser, the transducer vibrates the atomisation plate, causing it to oscillate in a forward-backward direction. During backward movement, the plate presses against the fluid retaining substrate, which causes disinfecting fluid to be pushed (at least partially) into the plurality of holes in the plate. During forward movement, this disinfecting fluid is ejected from the plate in a forward direction. The transducer may operate at about 100 kHz, such as about 108 kHz. The atomisation plate may have about 700 holes. The holes may be substantially circular. The holes may have a diameter of about 10 μm.

Any features discussed above in relation to the apparatus of the first aspect may be used in combination with the nebuliser assembly of the second aspect, and vice versa.

The fluid retaining substrate may act as store of disinfecting fluid immediately prior to atomisation. Although a reservoir of fluid could be placed in contact with the atomisation plate to achieve this, doing so may result in an inconsistent amount of fluid reaching different parts of the atomisation plate (e.g., if operated in different orientations) and/or would not allow for the amount of atomised fluid to be controlled. Therefore, it is particularly preferable to use a fluid retaining substrate to which fluid can be supplied to saturate it with different amounts of fluid.

Preferably, the fluid retaining substrate comprises a sponge material. Advantageously, a sponge material is able to store disinfecting fluid substantially evenly throughout its volume. Furthermore, a sponge material is able to accommodate a range of moisture levels depending on the amount of fluid that is supplied. The sponge material may be made of polyurethane. The fluid retaining substrate preferably comprises a layer of material. The layer may be substantially circular. The layer may have a surface that is parallel to (and preferably substantially covering) the back surface of the atomisation plate.

The nebuliser assembly may further comprise a moisture sensor arranged to detect moisture in the fluid retaining substrate. In this way, it is possible to detect whether fluid is supplied to the fluid retaining substrate.

Preferably, the moisture sensor is a binary sensor configured to detect the presence or absence of disinfecting fluid in the fluid retaining substrate. In this way, the moisture sensor can be relatively simple and inexpensive while still enabling detection of faults in the apparatus (such as inadequate operation of a fluid pump).

Preferably, the moisture sensor comprises a first electrode and a second electrode in contact with the fluid retaining substrate, and the moisture sensor is configured to detect electrical current between the electrodes. When the fluid retaining substrate is substantially dry, then no substantial current is detected between the electrodes. On the other hand, where disinfecting fluid is present within the fluid retaining substrate, electrical current is able to flow between the electrodes through the fluid retaining substrate. Therefore, by monitoring the current (such as with a controller in the apparatus), it is possible to detect a fault. The controller may be part of the nebuliser assembly. Alternatively, the controller may be provided in a separate part of the disinfecting apparatus.

Alternatively, the moisture sensor may provide a measurement of the amount of disinfecting fluid within the fluid retaining substrate. In this instance, the moisture sensor may be configured to transmit measurements to a controller of the disinfecting apparatus to enable adjustment of the flow of disinfecting fluid to the nebuliser assembly.

By transmitting moisture measurements to a controller, the controller can adjust the amount of disinfecting fluid that is supplied to optimize operation of the apparatus. For example, the nebuliser assembly may operate most efficiently when the moisture levels in the fluid retaining substrate are at a particular level (e.g., a particular level of saturation). Therefore, if the moisture sensor detects a deviation from this particular level, the controller may adjust the amount of disinfecting fluid that is provided from a reservoir (e.g., of the disinfecting apparatus) so as to restore the moisture levels in the fluid retaining substrate to the particular level. The controller may use closed loop feedback.

The nebuliser assembly may further comprise an attachment system to enable the nebuliser assembly to be releasably attached to the disinfecting apparatus. In this way, the nebuliser assembly may be easily replaced and repaired by removing it from the disinfecting apparatus.

Preferably, the attachment system comprises a magnetic attachment system. Preferably, the magnetic attachment system comprises a plurality of magnets. Preferably, four magnets are used. Alternatively, a different number of magnets may be used. By using a plurality of magnets, the nebuliser assembly can be retained in a predetermined position and orientation on the disinfecting apparatus. Alternatively, a single magnet may be used. The plurality of magnets may be provided on the nebuliser assembly so that they align with a corresponding magnet or magnetic material on the apparatus. Alternatively, a plurality of magnets may be provided on the apparatus, so that they align with corresponding magnets or magnetic material on the nebuliser assembly. Therefore, the magnetic attachment system of the nebuliser assembly does not necessarily require magnets. Alternatively or additionally, the attachment system may include hooks, clips or any other suitable means for attaching the nebuliser assembly to the disinfecting apparatus.

The nebuliser assembly may further comprise a casing that at least partially encloses the fluid retaining substrate. In this way, leakage from the nebuliser assembly can be inhibited by the casing. The casing may have at least one opening to enable disinfecting fluid to be supplied to the fluid retaining substrate. For example, the casing may comprise a back opening for receiving fluid from a fluid reservoir of the apparatus. The casing may have at least one front opening in which each of the nebulisers are positioned.

The plurality of magnets may be provided inside the casing. The casing may have a material and/or thickness that does not substantially inhibit operation of the magnetic attachment system. For example, the casing may comprise plastic. Preferably, the fluid retaining substrate is compressed at least partially within the nebuliser assembly. The casing may be configured to at least partially compress the fluid retaining substrate against the atomisation plate. For example, the fluid retaining substrate may have a thickness that is greater than a distance between the back surface of the atomisation plate and an opposing surface of the casing; in this way, the fluid retaining substrate is compressed during use. Advantageously, maintaining partial (but not complete) compression of the substrate improves consistency of contact between the atomisation plate and the fluid retaining substrate, thereby resulting in more consistent atomisation.

The nebuliser assembly may comprise an opening that is aligned with the position of the fluid retaining substrate, such that the fluid can be supplied to the fluid retaining substrate via the opening. In this way, the amount of fluid retained in the substrate can be adjusted responsively by adding fluid to the opening and/or adjusting a rate of fluid supply. For example, the saturation level of disinfecting fluid in the substrate can be adjusted by adding fluid through the opening. Therefore, the openings allow for improved control (e.g., active control) over the amount of fluid that is stored in the nebuliser and therefore atomised by the nebuliser for dispersion throughout a space.

When the nebuliser assembly is attached to a disinfecting apparatus (e.g., using the magnetic attachment system or otherwise), the opening is configured to align with a fluid supply within the disinfecting apparatus. For example, during use of the apparatus, a pump in the apparatus may supply fluid to the opening thereby controlling the amount of fluid retained in the fluid retaining substrate.

Preferably, the nebuliser assembly comprises one or more additional nebulisers, each comprising a corresponding atomisation plate, and transducer. Any of the features described in relation to the (e.g., first) nebuliser may apply to any of the one or more additional nebulisers. Preferably, there are a total of four nebulisers in the nebuliser assembly. Alternatively, a different number may be used, such as one, two, three, five or more. A single moisture sensor may be used to detect moisture within one of the fluid retaining substrates. Alternatively, more than one moisture sensor may be used. A fluid retaining substrate may be used for each of the additional nebulisers. Alternatively, a fluid retaining substrate may be used for two or more of the nebulisers.

Where there is a plurality of fluid retaining substrates, there may be a plurality of openings that each correspond (e.g., align with) a respective fluid retaining substrate. In this way, fluid may be supplied substantially uniformly to each of the fluid retaining substrates via the respective openings, thereby ensure that a consistent amount of fluid is atomised by each of the nebulisers. Alternatively, there may more or fewer openings than the number of fluid retaining substrates. In one example, a single opening may be used to supply fluid to all of the fluid retaining substrates, which may simplify the construction of the nebuliser assembly and/or the disinfecting apparatus.

Also described herein is a disinfecting apparatus comprising the nebuliser assembly as described above and herein.

According to a third aspect of the present invention, there is provided an apparatus for disinfecting a space, comprising: a reservoir for containing disinfecting fluid; a nebuliser fluidly connected to the reservoir, the nebuliser configured to atomise the disinfecting fluid; and a fan arranged to receive air from one or more input vents at a base of the disinfecting apparatus and direct the air through one or more output vents arranged adjacent to the nebuliser, thereby dispersing the atomised fluid through the space.

Advantageously, the airflow from the output vents helps to increase the dispersion of the atomised fluid throughout the space. By providing the input vents towards a base of the apparatus, the air they receive is less likely to have reached the input vents directly from the output vents. This helps to ensure better circulation of atomised disinfecting fluid in the space and enables more accurate measurements of properties of the air (e.g., humidity). Any of the features described above in relation to the apparatus of the first aspect and/or the nebuliser assembly of the second aspect can be provided in combination with the apparatus of the third aspect, and vice versa. Preferably, the reservoir is the primary reservoir. However, it will be appreciated that the apparatus may only have a single reservoir.

Preferably, the fan is a centrifugal fan. Advantageously, a centrifugal fan is a simple and cost-effective component, thereby reducing the time and cost associated with manufacturing the apparatus.

Preferably, the input vents comprise a plurality of slots arranged at least partially around a perimeter of the base. The plurality of slots may include at least one slot provided adjacent a front surface of the apparatus. Preferably, at least one slot is provided adjacent a side and/or a back surface of the apparatus. In this way, air circulation within the space is improved, thereby improving disinfection levels. Furthermore, the air received into the input vents is less likely to have been received directly from the output vents and/or the nebulisers.

Preferably, the input vents include a plurality of openings arranged in a grille on an underside of the base. Advantageously, this may increase the maximum airflow rate that can be supplied to the fan. Preferably, the base of the apparatus is spaced from the floor. The base may comprise one or more supports, such as support feet. The base may be substantially rectangular. The supports are preferably provided at each corner of the base.

The apparatus may further comprise a humidity sensor arranged to receive air from the one or more input vents. Advantageously, this enables progress of a disinfecting cycle to be monitored by the apparatus. Based on this monitoring, the disinfecting cycle may be adjusted to ensure that adequate disinfection is being applied by the apparatus. Since the humidity receives ambient air through the one or more input vents, the apparatus can determine the amount of disinfecting fluid in the air (e.g., a humidity level and/or amount of saturation). The humidity sensor is preferably located in an airflow pathway between the input vents and the output vents. The humidity sensor may be provided prior to the fan, after the fan, or as part of the fan.

The apparatus may further comprise a controller configured to receive measurements from the humidity sensor and adjust operation of the apparatus based on the received measurements. In this way, a disinfection cycle can be adjusted to ensure that the space is being adequately disinfected. For example, the controller may maintain a specific humidity level (based on measurements by the sensor) for a certain period of time. Alternatively or additionally, the humidity sensor may turn off the nebuliser once a certain humidity level is reached.

Preferably, the controller is configured to stop operation of the nebuliser when a threshold relative humidity is detected by the humidity sensor, preferably wherein the threshold relative humidity is about 92%. Advantageously, the threshold relative humidity level may indicate that the disinfecting fluid will condense on surfaces in the space. It has been found that 92% relative humidity corresponds to condensation of the disinfecting fluid. Alternatively, other thresholds may be used, such as about 90% to about 95%.

Preferably, at least one of the one or more output vents is located between the nebuliser and the input vents. Advantageously, this arrangement improves the dispersion of atomised droplets throughout the space, since the output airflow may assist in carrying the droplets away from the apparatus (e.g., rather than directing droplets towards an input airflow in the opposite direction). In this context, the term “between” may refer to the relative arrangement of the output vents, input vents and nebuliser on an external surface of the apparatus (e.g., a relative arrangement in a vertical direction). For example, where the input vents are located at or adjacent the base, the nebuliser is located at a larger height from the base than the output vents.

Preferably, the one or more output vents are located below the nebuliser. In this way, the fluid may be more effectively dispersed throughout the space, since the atomised droplets are prevented from falling directly downwards from the nebuliser. For example, the air that is output from the output vents may help to carry atomised droplets further away from the apparatus rather than falling under the influence of gravity, thereby enhancing the dispersion of the disinfecting fluid. In one example, the input and output vents may be configured to establish a convection cycle where outgoing droplets are directed upward, and returning air is drawn in across a floor of the space.

This placement of the output vents is particularly advantageous when used together with the humidity sensor receiving air from the input vents; since movement of droplets directly from the nebuliser to the input vents is inhibited by the airflow from the output vents, readings taken by the humidity sensor are more likely to accurately correspond to the humidity of ambient air within the space (e.g., rather than local air containing droplets that had not been fully dispersed through the space).

It will be understood by a skilled person that any apparatus feature described herein may be provided as a method feature, and vice versa. It will also be understood that particular combinations of the various features described and defined in relation to any of the aspects herein can be implemented and/or supplied and/or used independently.

Moreover, it will be understood that the present invention is described purely by way of example, and modifications of detail can be made within the scope of the invention.

### BRIEF DESCRIPTION OF DRAWINGS

One or more embodiments of the present invention will now be described with reference to the accompanying figures, in which:

FIGS. 1A to 1C show perspective, exploded and cutaway view of a disinfecting apparatus having a primary reservoir and a secondary reservoir;

FIGS. 2A and 2B show a secondary reservoir that may be used as part of the apparatus;

FIGS. 3A and 3B show the connection interface between the primary and secondary reservoirs;

FIG. 4 shows a sensor to detect fluid in the primary reservoir;

FIGS. 5A and 5B show components of a nebuliser to atomise fluid that is supplied from the primary reservoir;

FIGS. 6A to 6C show different view of a nebuliser assembly having a plurality of the nebulisers shown in FIGS. 5A and 5B;

FIGS. 7A and 7B show cutaway and cross-sectional views of the apparatus so that the fan and corresponding air pathways are visible; and

FIG. 8 shows a view of the apparatus where the nebuliser assembly and an interface have been removed to show internal components.

### DETAILED DESCRIPTION

As discussed above, there is a need for a compact disinfection apparatus that enables control over the disinfection process, and which can operate reliably and safely.

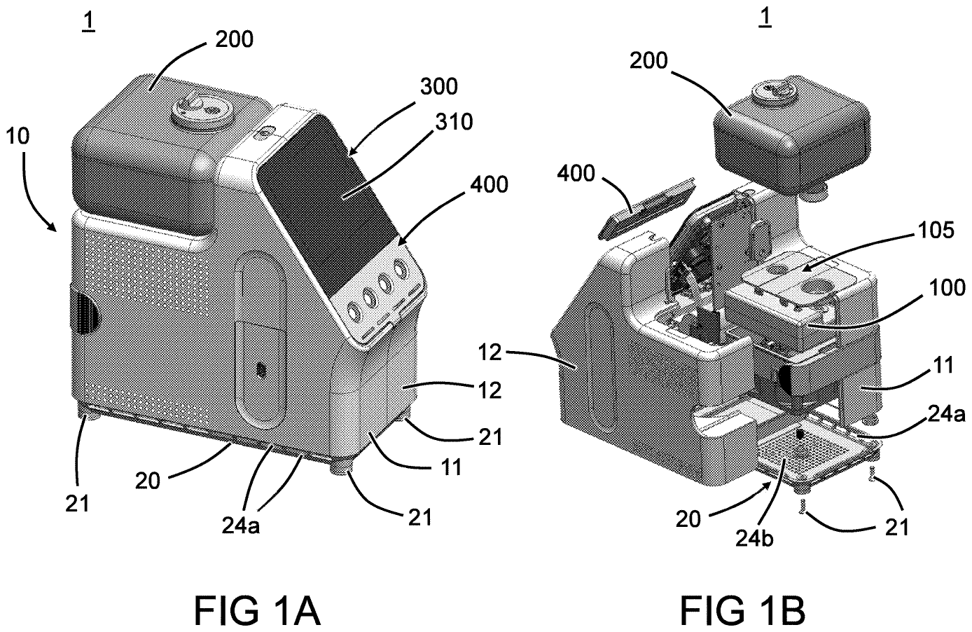

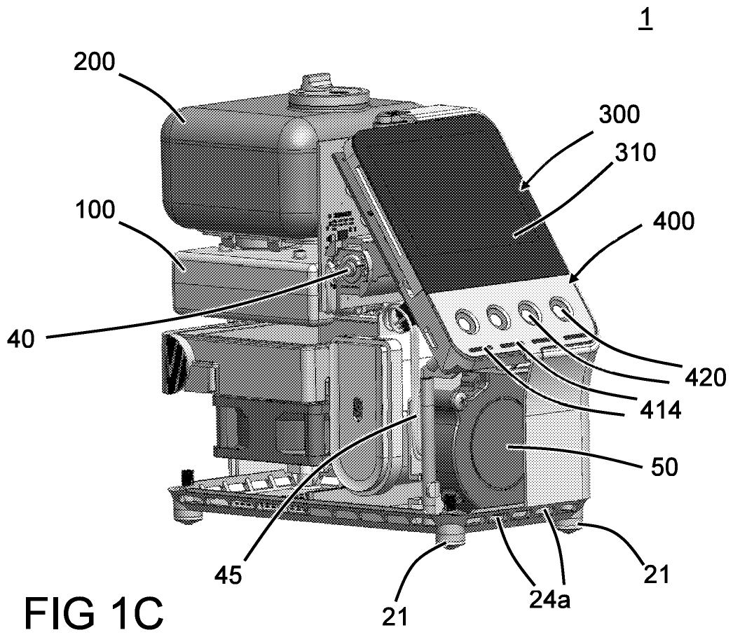

An example of such an apparatus 1 will now be described in detail. FIG. 1A shows a perspective view of the apparatus 1, and FIGS. 1B and 1C show exploded and cutaway views so that internal components are visible.

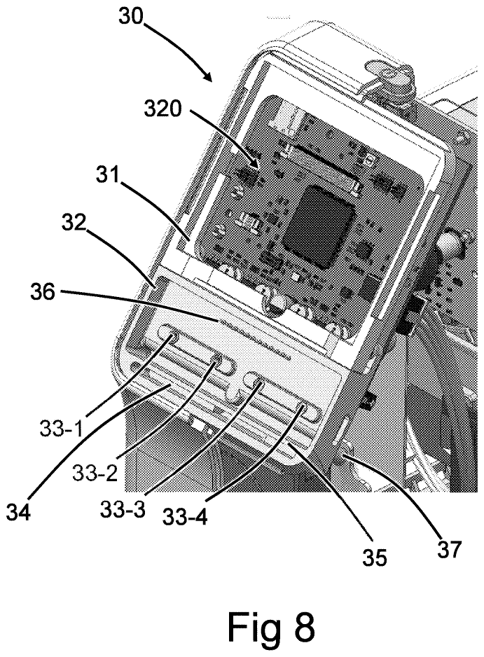

To summarize operation of the apparatus 1, a pump 40 transfers a disinfecting fluid (specifically hydrogen peroxide) from a pair of reservoirs 100, 200 through fluid channels 45 to a nebulizer assembly 400 arranged on a front panel 30 of the apparatus 1. When the fluid reaches the nebulizer assembly 400, a plurality of nebulizers 420 atomises the fluid so that it can be dispersed into the air within a space (e.g., a room, vehicle, or chamber in a separate apparatus). The dispersion of the atomised fluid is assisted using a fan 50 which directs air out of openings 414 in the nebuliser assembly 400. In this way, the air containing evaporated disinfecting fluid is distributed more evenly throughout the space so that it can condense and disinfect surfaces within the space. The front panel 30 of the apparatus 1 includes an interface 300 so that a user can instruct the apparatus 1 to carry out a particular disinfection cycle and/or receive data from the apparatus 1. To achieve this the interface 300 includes a screen 310. The apparatus 1 also includes a controller 320 to control operation of the apparatus 1 (see FIG. 8), such as to cause the pump 40 fan 50 and nebulizer assembly 400 to carry out a disinfection cycle. Further details of these components will now be described in detail.

As particularly shown in FIGS. 1A and 1B, the apparatus 1 has a housing 10, which includes panels 11, 12. When viewed from the front surface (one which the nebuliser assembly 400 is located), these panels may be referred to as a left panel 11 and a right panel 12. These panels 11, 12 cover both sides of the apparatus 1, as well as the front and back surfaces. At least one of the panels, (more specifically the right panel 12) may be removable from the apparatus 1 to enable replacement and/or repair of internal components. The housing 10 includes a base 20 to support the internal components. The base 20 is substantially rectangular, with support feet 21 arranged at each corner so as to space the base 20 from a surface on which it rests. The base 20 is attached to other parts of the apparatus 1 (e.g., the side panels 11, 12) with connectors 22. Here, the connectors 22 are bolts, though it will be appreciated that any suitable type of connector may be used for this purpose. The base 20 has a plurality of openings 24 (or “input vents”) to enable airflow into the apparatus 1. These openings 24 include slots 24a around the perimeter of the base 20 (only some of which are labelled, for clarity). These input vents 24 also include a plurality of openings arranged in a grille 24b on the underside of the base 20.

One problem with existing disinfection apparatuses is the difficulty in ensuring that they always have sufficient disinfection fluid to fully carry out a disinfection cycle. This is because typical disinfection apparatuses have a single reservoir. The single reservoir is either an integrated refillable reservoir, or a replaceable reservoir (which may be referred to as a cartridge). Users tend to prefer the cartridge-based approach, since this significantly reduces the time and expertise required to refill the apparatus. However, it is not straightforward to determine how much fluid is remaining inside the reservoir, which can lead to a disinfection cycle being initiated without there being sufficient fluid to complete the cycle. Unless a cycle is fully completed, it is not safe for human operators to re-enter the space to refill the apparatus with disinfecting fluid. In view of this, an improved approach is to provide sensors in the reservoir so that the cycle does not begin when there is insufficient fluid. However, such sensors increase the cost and complexity of the reservoir, making in unsuitable for a solely cartridge-based approach.

To address the above, the apparatus 1 provides a particularly advantageous configuration, where two reservoirs 100, 200 are present. A primary reservoir 100 is positioned inside the housing 20. The primary reservoir 100 may be referred to as a “first reservoir” or an “integrated” reservoir. A secondary reservoir 200 is removably attached to the primary reservoir 100, via a connection interface on an upper surface of the primary reservoir 100. The secondary reservoir 200 may be referred to as a “second reservoir” or a “cartridge”.

The primary reservoir 100 is configured to supply the disinfecting fluid to the nebulizer assembly 400 during use. To do so, the primary reservoir 100 is connected to the nebulizer assembly 400 via the pump 40 and one or more fluid channels 45 (as particularly shown in FIG. 1C). The primary reservoir 100 has a volume that is sufficient to contain all the fluid required for a complete disinfection cycle. Depending on the type of disinfection cycle being performed, the amount of disinfecting fluid will be between about 60 mL and about 120 mL. Therefore, the volume of the primary reservoir 100 is about 120 mL.

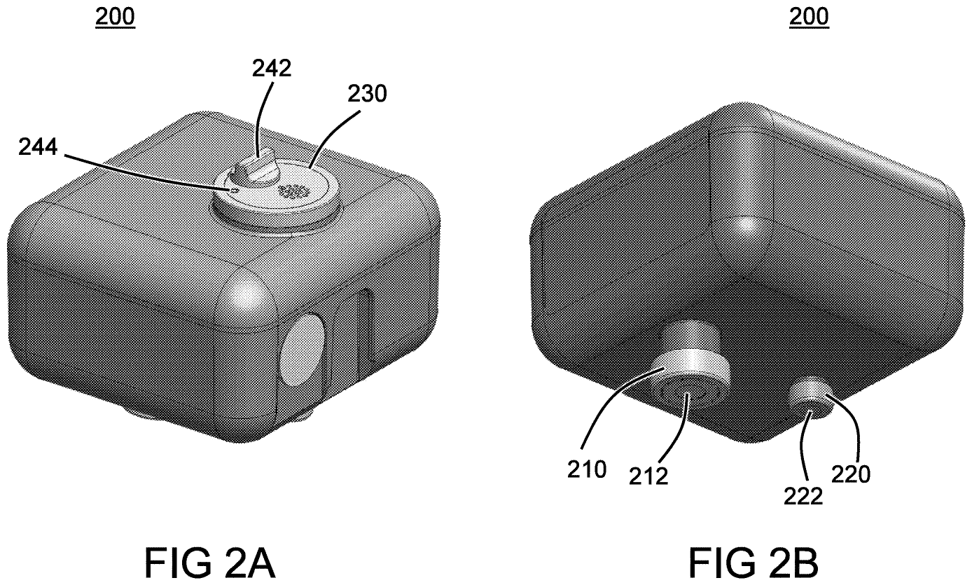

The secondary reservoir 200 is shown in FIGS. 2A and 2B. The secondary reservoir 200 is used to replenish fluid in the primary reservoir 100, thereby allowing several disinfection cycles to be performed before replacing the secondary reservoir 200. In this example, the secondary reservoir 200 has a volume of 270 mL. Since the secondary reservoir 200 is removable and replaceable, the apparatus 1 can be refilled with fluid quickly and easily. Therefore, by using both the primary reservoir 100 and secondary reservoir in combination, it is possible to ensure that disinfection cycles are reliably completed without increasing the cost and/or complexity of the components, and without increasing the difficulty of refilling the apparatus 1.

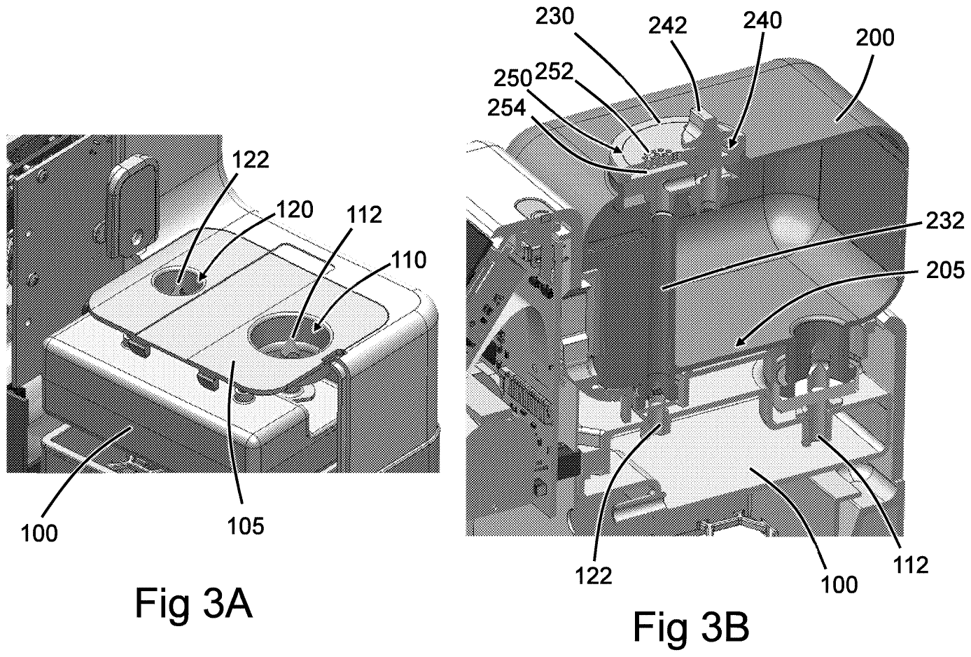

The connection between the primary reservoir 100 and secondary reservoir 200 will now be described with particular reference to FIGS. 3A and 3B. The primary reservoir 100 has a first connector 110 and a second connector 120 arranged on its top surface. These connectors 110, 120 may be considered as providing a connection interface 105 of the primary reservoir 100. The secondary reservoir 200 has a first port 210 and a second port 220 arranged on its bottom surface at positions corresponding to the positions of the first and second connector 110, 120. These ports 210, 220 may be considered as providing a connection interface 205 of the secondary reservoir 200.

The first connector 110 comprises a first needle 112, and the second connector 120 comprises a second needle 122. Each needle 112, 122 is configured to extend into a respective first and second membrane 212, 222 of the first and second ports 210, 220, thereby establishing fluid communication between the reservoirs 100, 200. While the membranes 212, 222 can be opened using the needles 112, 122, they are configured to close back on themselves once the needles 112, 122 are removed. This enables separate storage of the secondary reservoir 200 between uses of the apparatus 1. To facilitate this, the membranes 212, 222 are manufactured from a flexible material such as PTFE or silicon.

The first connector 110 (and first port 210) is configured to enable flow of disinfecting fluid from the secondary reservoir 200 to the primary reservoir 100. Thus, the first needle 112 may have an internal diameter between 5.5 mm and 10 mm, since this is sufficient for fluid to flow easily, but not so large that air can bubble through the needle 112.

The second connector 120 (and second port 220) is configured to enable flow of displaced air from the primary reservoir 100 to the secondary reservoir 200. Thus, the second needle 122 may have an internal diameter less than 5.5 mm; in this example the second needle 122 has an internal diameter of 3 mm, since this allows for sufficient airflow but does not allow for flow of fluid through the second connector 120.

During use of the apparatus 1, disinfecting fluid is pumped out of the primary reservoir 100 by the pump 40 and replenished with fluid from the secondary reservoir 200. Therefore, it is beneficial to enable airflow into the secondary reservoir 200 and/or the primary reservoir 100 during use, in order to replace the fluid being used. Additionally, due to changes in atmospheric conditions (e.g., temperature and pressure), it is beneficial for the pressure in the primary reservoir 100 and/or secondary reservoir 200 to equilibrate with the surrounding ambient air. To address these challenges, the secondary reservoir 200 includes a valve assembly 230. The valve assembly 230 is connected to the second port 220 via an air channel 232. The air channel 232 enables air entering the secondary reservoir 200 from the second port 220 to be supplied directly to the valve assembly 230 without needing to bubble through fluid in the secondary reservoir 200.

However, is not straightforward to provide a valve assembly 230 that is capable of allowing large volumes of air to flow (e.g., during use of the apparatus 1), as well as allowing smaller volumes to flow (e.g., due to changes in atmospheric conditions), while also preventing leakages of the fluid from the secondary reservoir 200. In view of this, the valve assembly 230 includes a first valve 240 and a second valve 250.

The first valve 240 includes a plug 242 that is rotatable within the valve assembly 230 between a first position where airflow between the primary reservoir 100 and the secondary reservoir 200 is inhibited, and a second position where airflow between the primary reservoir 100 and the secondary reservoir 200 is enabled. Therefore, by moving the plug 242 to the first position, it is possible to prevent fluid from emptying from the secondary reservoir 200 to the primary reservoir 100. Conversely, by moving the plug 242 to the second position, airflow from the primary reservoir 100 to the secondary reservoir 200 is enabled, thereby allowing fluid to flow from the secondary reservoir 200 into the primary reservoir 100. Prior to initiation of a disinfection cycle, and operator may rotate the plug 242 to its second position, which enables larger volumes of air to enter the secondary reservoir 200 (e.g., to replace the fluid that is consumed during the disinfection cycle). In the second position of the plug 242, an airflow pathway is established between a vent 244 on the valve assembly 230 and the interior of the secondary reservoir 200. In FIG. 3B, the plug 242 is shown in its second position, where airflow between the primary reservoir 100 and secondary reservoir 200 is enabled (via the air channel 232), as well as airflow to the surrounding environment (via second valve 250 and/or the vent 244). In this position, air is able to flow into the apparatus 1 through the valve assembly 230 and between the secondary reservoir 200 and the primary reservoir 100 (through the air channel 232).

The second valve 250 includes a plurality of holes 252 and a semi-permeable membrane 254. The membrane 254 is at least partially permeable to air but is substantially impermeable to fluid. The membrane 254 may be a breathable hydrophobic membrane 254. For example, Goretex® may be used for the membrane 254. In this way, even when the plug 242 of the first valve 240 is in the first position, air may flow into and/or out of the secondary reservoir 200 so as to equalise the internal pressure with ambient pressure.

As mentioned above, initiation of a disinfection cycle may be prevented when there is insufficient fluid within the primary reservoir 100. In order to determine whether this is the case, the apparatus 1 includes a sensor 130. The sensor 130 may be implemented in several ways. For example, the sensor 130 could be configured to provide an estimate for the amount of fluid remaining in the primary reservoir 100 (either as a percentage or a specific volume). However, in this example, the sensor 130 is a binary sensor that indicates that either sufficient fluid is present, or that insufficient fluid is present. As a result, the cost and complexity of the sensor 130 can be significantly reduced.

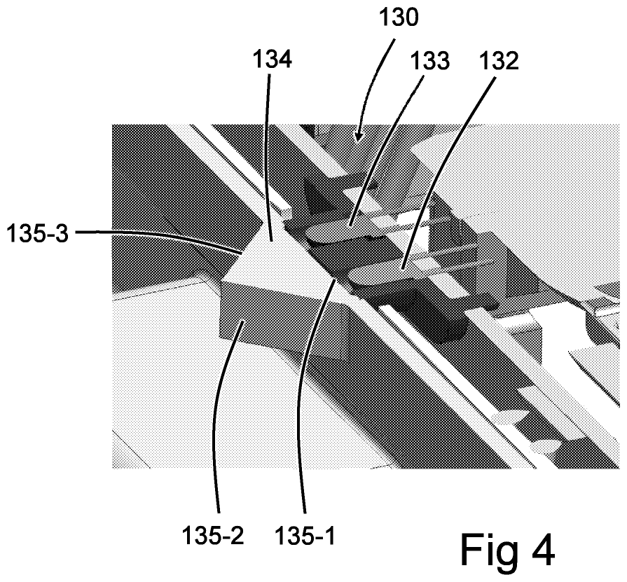

The sensor 130 is shown in detail in FIG. 4. The sensor 130 includes an optical source 132 and an optical sensor 133, which are arranged side-by-side adjacent an external surface of the primary reservoir 100. In this example, the optical source 132 and the optical sensor 133 are not themselves part of the primary reservoir 100, though in other examples, such components can be included as part of the primary reservoir 100. The primary reservoir 100 includes a transparent member in the shape of a prism 134. The prism 134 includes a first face 135-1 that forms part of an external surface of the primary reservoir 100. The prism includes a second face 135-2 and a third face 135-3 that form part of an internal surface of the primary reservoir 100. The sensor 130 is positioned towards the top of the primary reservoir 100 so that the second and third faces are only in contact with the disinfecting fluid when the primary reservoir 100 is substantially full. The prism 134 may be made of glass. Alternatively, other transparent materials (e.g., a plastic such as acrylic) may be used. The angle between the second face 135-2 and third face 135-2 is 90°. The angle between the first face 135-1 and each of the second face 135-2 and third face 135-3 is 45°. Thus, the prism 134 has a cross-section in the shape of a right-angled isosceles triangle.

The sensor 130 may operate as follows. First, the optical source 132 is activated, thereby emitting light that enters the prism 134 perpendicular to the first face 135-1. This light hits the second face 135-2 at an angle of incidence of 45°. The path of the light subsequently depends on the critical angle at the boundary of the prism 134. The critical angle is θ_{c}=arcsin(nᵣ/nₚ), where nᵣ is the refractive index in the reservoir, and nₚ is the refractive index in the prism 134.

If the prism 134 is not submerged in fluid, then nᵣ≈1 and nₚ=1.59 (for a polycarbonate prism), giving a critical angle of about 39°. Therefore, since the angle of incidence (45°) is greater than this critical angle, the light totally internally reflects at the second face 135-2. For the same reason, the light also totally internally reflects at the third face 135-3, and exits the prism 135 through the first face 135-1 to reach the optical sensor 133.

On the other hand, when the prism 134 is submerged in fluid (e.g., hydrogen peroxide having a refractive index of about 1.39), then the critical angle is about 61°. Therefore, since the angle of incidence (45°) is smaller than this critical angle, the light from the optical source 132 passes through the second face 135-2 without significant reflection, and no substantial light is detected by the optical sensor 133.

Thus, based on the output of the optical sensor 133, it is possible to determine whether the prism 134 is submerged in disinfecting fluid or not. While specific materials and angles have been used in the example above, alternative shapes and materials of the prism may be used provided that the optical sensor 133 provides different outputs based on whether the prism 134 is submerged in the disinfecting fluid. For example, the prism 134 may instead be a material such as glass. Furthermore, different wavelengths of light may be used (e.g., infrared, visible light, and/or ultraviolet).

The sensor 130 may be connected to the controller 320 in order to operate the optical source 132 and receive measurements from the optical sensor 133. When the apparatus 1 is instructed to carry out a disinfection cycle, the controller 320 may activate the optical source 132 and receive a measurement from the optical sensor 133. If the controller 320 determines that the primary reservoir 100 is not full of disinfection fluid, then initiation of the disinfection cycle is prevented. An alert may be sent to the user to indicate that the primary reservoir 100 should be refilled (e.g., by replacing the secondary reservoir 200). If the controller 320 determines that the primary reservoir 100 is substantially full, then the disinfection cycle proceeds. Since the volume of the primary reservoir 100 is sufficient to allow completion of the disinfection cycle, there is no risk of running out of fluid partway through the cycle.

Although not shown in the Figures, the secondary reservoir 200 comprises a writable RFID tag. When the secondary reservoir 200 is filled (either by an operator or when the secondary reservoir 200 is manufactured and shipped to the user), the volume of fluid contained in the secondary reservoir 200 is recorded on the RFID tag. The apparatus 1 includes an RFID reader (not shown) that is able to communicate with the RFID tag when the secondary reservoir 200 is attached to the apparatus 1. In this way, the RFID tag can be updated with an estimate of the volume remaining. For example, the pump 40 may operate at a known volume flow rate during the disinfection cycle and/or a particular disinfection cycle may use a predetermined amount of fluid. The volume of fluid consumed can be subtracted from the value stored on the RFID tag prior to a disinfection cycle. In this way, the operator can be kept informed of the approximate amount of fluid left within the secondary reservoir 200, such as by an alert or message on the interface 300. By providing the estimate with an RFID tag, different secondary reservoirs 200 can be used interchangeably with different apparatuses 1, while still ensuring that the volume estimate provided is accurate.

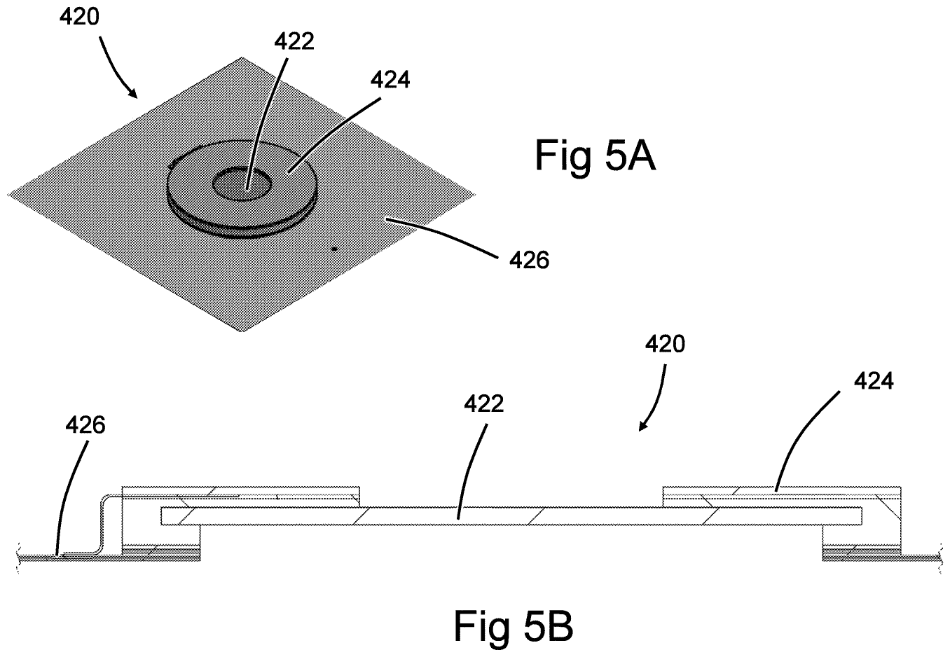

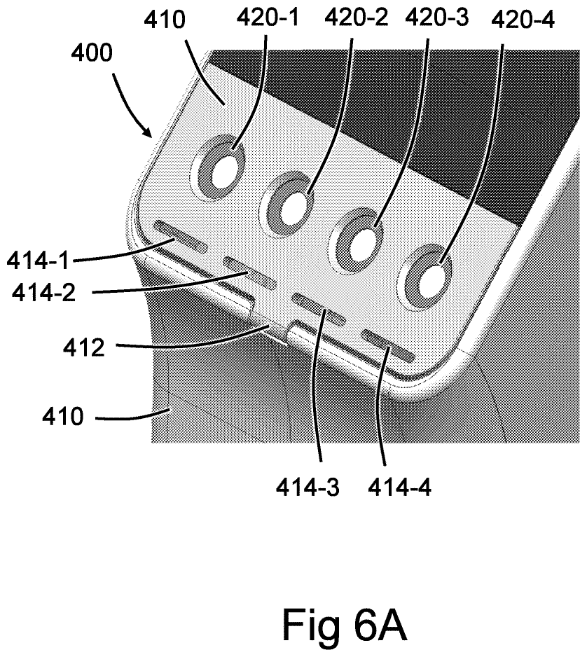

The nebuliser assembly 400 will now be described in detail. As mentioned previously, the nebuliser assembly 400 includes a plurality of nebulisers 420. One of these nebulisers 420 is shown in more detail in FIGS. 5A and 5B. FIG. 5A shows a perspective view of the nebuliser 420 and FIG. 5B shows a cross section of the nebuliser 420. The nebuliser 420 has a fluid excitation portion 422, and transducer 424. The fluid excitation portion 422 is substantially circular, and the transducer 424 is substantially ring-shaped. The transducer 424 induces vibrations in the fluid excitation portion 422 so as to atomise fluid that is supplied to the nebuliser 420. The transducer 424 induces vibrations at about 108 kHz. The fluid excitation portion 422 is a flat (e.g., planar) sheet of material, such as a ceramic sheet or a metal sheet. The fluid excitation portion 422 may be referred to herein as an atomisation plate 422. The atomisation plate 422 has a front surface (the top surface as shown in FIG. 5B) and a back surface (i.e., the bottom surface as shown in FIG. 5B). While not visible in FIG. 5, the fluid excitation portion 422 has a plurality of holes in the form of a mesh. The plurality of holes extend between the front surface and back surface of the atomisation plate 422 so that fluid can pass therethrough. More specifically, about 700 holes are present in this mesh. The holes are substantially circular and have a diameter of about 10 μm. The plurality of holes enables fluid supplied to the nebuliser 420 to pass through the atomisation plate 422 and generate droplets via vibrations transmitted to the atomisation plate 422 by the transducer 424.

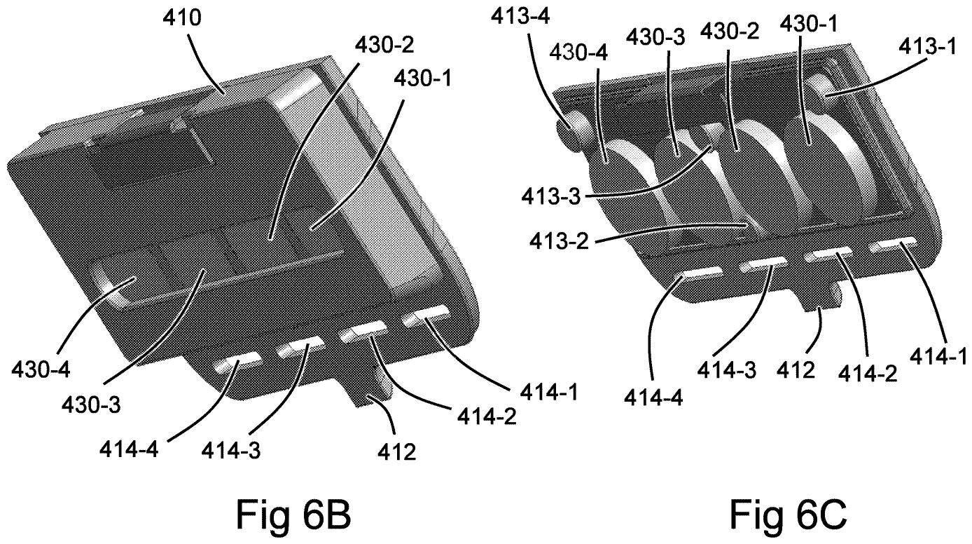

The transducer 424 and atomisation plate 422 are arranged on a flexible substrate 426, which may be a PCB. As shown in FIG. 6B, the flexible substrate 426 may provide an electrical connection to the transducer 424 so that it can be controlled to induce vibrations in the atomisation plate 422. Further details of the nebuliser 420 are disclosed in EP Pub. No. 4000748A1, titled “Injection Head for Excitation of Fluid” and published May 25, 2022.

The other components of the nebuliser assembly 400 will now be described in detail with reference to FIGS. 6A to 6C. FIG. 6A shows the nebuliser assembly 400 in its position on the front surface of the apparatus 1. The nebuliser assembly 400 has a casing 410. The nebuliser assembly 400 may be removable from the apparatus 1, as shown in FIG. 6B. To enable reliable connection with rest of the apparatus 1, the casing 410 may have an attachment system. The attachment system includes a protrusion 412 to ensure that the nebuliser assembly 400 can only be installed at a predetermined position on the apparatus 1. As shown in FIG. 6A, the protrusion 412 fits within a corresponding slot in the housing 10 when the nebuliser assembly 400 is installed. Other components may be used in the attachment system, such as a magnetic attachment system. This allows the nebuliser assembly 400 to be easily removed and reattached to the front surface of the apparatus 1, such for maintenance, repair and/or replacement. As shown in FIG. 6C, the magnetic attachment system includes four magnets 413. While not shown in the Figures, these magnets 413 align with corresponding magnets in the housing 10 of the apparatus 1. Advantageously, by using a magnetic attachment system, the magnets 413 can be covered by other parts of the nebuliser assembly 400 such as the casing 410 (see FIG. 6B) while still allowing them to secure the nebuliser assembly 400 within the apparatus 1.

As shown in FIG. 6A, four nebulisers 420 are provided on a front surface of the casing 410. The nebulisers 420 are substantially identical to each other and correspond to the nebuliser described above in relation to FIGS. 5A and 5B. The nebulisers 420 are located side-by-side in a linear arrangement. Below each of the nebulisers 420 a corresponding opening 414 (or “output vent”) is provided in the casing 410. Each of these output vents 414 is an elongate horizontal slot 414. As described later, airflow from the fan 50 is directed through these output vents 414 during a disinfection cycle, thereby dispersing atomised disinfecting fluid throughout the space. The length of each elongate slot 414 substantially corresponds to the diameter of each nebuliser 420.

As shown particularly in the cutaway view in FIG. 6C, the nebuliser assembly 400 comprises a sponge 430 corresponding to each of the nebulisers 420. The sponge 430 is made of polyurethane. The (each) sponge 430 is arranged to contact the back of each of the nebulisers 420 so that during vibration of the fluid excitation portion 422, the fluid excitation portion 422 periodically presses into the sponge 430. In this way, disinfecting fluid retained in the sponge 430 can be forced into the holes in the fluid excitation portion 422 (e.g., during backward motion) and then ejected from the nebuliser 420 (e.g., during forward motion). By using a sponge 430 to supply the fluid to the nebuliser 420 (i.e., instead of a reservoir of fluid contacting the nebuliser 420) it is possible to control the amount of fluid that is ejected, such as by altering the saturation level of disinfecting fluid within the sponge 430. For example, if a larger volume of fluid is desired within the space, the amount of fluid supplied to each sponge 430 may be increased. In this way, the nebuliser 420 can be provided with sufficient disinfecting fluid, while preventing leakage from the apparatus 1 (due to oversaturation of the sponge 430).

While not shown in FIG. 6, a moisture sensor may be present within the nebulizer assembly to detect presence of moisture within the (or each) sponge 430. A moisture sensor may be present in each sponge 430, or fewer moisture sensors may be used, such as only a single moisture sensor in one of the sponges. More specifically, two electrodes are provided in contact with one of the sponges 430. When the sponge 430 contains fluid, current can be detected between the electrodes. Thus, the moisture sensor is a binary sensor that indicates whether fluid is present in the sponge 430 or not. This type of sensor is particularly easy to manufacture and operate and thus does not substantially increase the cost of the apparatus 1 while still enabling useful measurements to be collected. For example, the electrodes may be connected to the controller 320 so that operation of the apparatus 1 can be adjusted based on the measurements by the moisture sensor. For example, if the moisture sensor does not detect fluid even after operation of the pump, it may be determined that a fault is present in the apparatus 1 and an alert may be triggered.

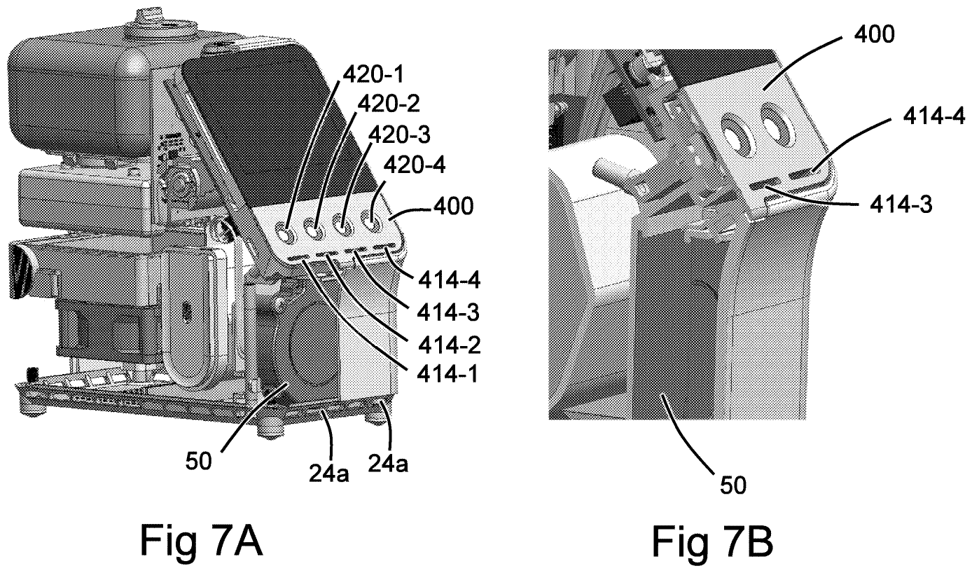

Operation of the fan 50 will now be discussed with reference to FIGS. 7A and 7B. As mentioned above, during a disinfection cycle (i.e., when the nebulisers 420 are active), air may be received into the apparatus 1 through input vents 24 (i.e., slots 24a around the edge of the base 20 and through the grille 24b on the underside of the base 20). The fan 50 directs this air to the nebuliser assembly 400 and out of the output vents 414. In this implementation, the fan 50 is a centrifugal fan 50. This type of fan 50 is relatively inexpensive, while still providing a sufficient airflow rate to disperse disinfecting fluid throughout the space.

In this example, the nebuliser assembly 400 has four output vents 414, each corresponding to one of the nebulisers 420. In particular, an output vent 414 is provided below each of the nebulisers 420 on the front surface of the nebuliser assembly 400. The output vents 414 are in the form of elongate slots 414 with a length substantially corresponding to the diameter of each nebuliser 420. In this way, the airflow out of the vents 414 prevents atomised droplets from falling directly downwards once generated at each nebuliser 420, so the distribution of atomised disinfecting fluid throughout the space is improved. In alternative examples, a different number of output vents 414 may be used, such as two larger vents, each corresponding to a pair of nebulisers 420.

While not shown in FIGS. 7A and 7B, the apparatus 1 also includes a humidity sensor arranged in an airflow path between the input vents 24 and the output vents 414. In this instance, the humidity sensor is located after the fan 50, though in other implementations, the humidity sensor may be located before the fan 50 or may be part of the fan 50. The humidity sensor can be used to monitor progress of a disinfection cycle, such as to indicate whether the amount of atomised disinfection fluid within the space has reached a sufficient level. The humidity sensor is connected to the controller 320. The controller 320 is configured to adjust operation of the apparatus 1 based on measurements by the humidity sensor. For example, controller 320 may operate the nebulisers 420 to maintain a specific humidity level for a certain period of time. Alternatively or additionally, the controller 320 may operate the nebulisers 420 until a threshold relative humidity is reached in the space, and subsequent turn off the nebulisers 420. It has been found that humidity levels of about 92% correspond to the point where hydrogen peroxide condenses within the space. Therefore, the controller 320 may operate the nebulisers 420 until a threshold relative humidity of 92% is reached.

In some implementations, the humidity sensor only detects the amount of disinfection fluid in the air. However, in this implementation, the humidity sensor detects presence of both the disinfection fluid (hydrogen peroxide) as well as water.

Since the humidity measurement ideally provides an indication of the amount of disinfecting fluid throughout the entire space (rather than only locally to the apparatus 1), it is advantageous to separate the airflow into the input vents 24 from the airflow away from the nebuliser 420. This is achieved by positioning the output vents 414 below each of the nebulisers 420 so that they are between the nebulisers 420 and the input vents 24. In this way, the air reaching the humidity sensor (via input vents 24) is more likely to correspond to the ambient air within the space. Additionally, the input vents 24 are not only provided on the front surface of the apparatus 1 but are provided by slots 24a all the way around the base 20, and additionally via the grille 24b on the underside of the base 20. Therefore, airflow reaching the humidity sensor can be kept substantially separate to airflow away from the apparatus 1.

FIG. 8 shows the front panel 30 of the apparatus 1 where the nebuliser assembly 400 and the interface 300 have been hidden. The front panel 30 has a first frame 31 that retains the interface 300 and a second frame 32 that retains the nebuliser assembly 400. As discussed above, the nebuliser assembly 400 may be removably attached to the second frame 32 using a magnetic attachment system. While not visible in FIG. 8, magnets are provided at positions corresponding to magnets 413 on the nebuliser assembly 400.

The second frame 32 is connected to the primary reservoir 100 by the conduits 45. The disinfecting fluid is supplied to the nebuliser assembly 400 via four openings 33 that align with the position of each of the sponges 430. In this way, a consistent amount of fluid can be supplied to all of the nebulisers 420. The second frame 32 also includes a drainage port 37 to allow for excess disinfecting fluid to be removed from the nebuliser assembly 400. Additionally, air is supplied to the second frame from the fan 50. Once the air reaches the second frame 32, it passes through an elongate slot 34 that aligns with the output vents 414 of the nebuliser assembly. The elongate slot 34 (supplying air) is separated from the openings 33 (supplying disinfecting fluid) by a partition wall 35. When the nebuliser assembly 400 is attached to the second frame 32, the partition wall 35 is arranged in sealing contact with the housing 410 of the nebuliser assembly 400 so that disinfecting fluid and air do not mix.

The second frame 32 also includes electrical connectors such as pins 36 that engage with corresponding connectors on the nebuliser assembly 400. In this way, power and/or control signals can be supplied to the nebulisers 420 (i.e., to the transducers 424). The power and/or control signals may be sent to the pins 36 from the controller 320. While in this example, the controller 320 is located behind the screen 310, it will be appreciated that the controller 320 can be located anywhere in the apparatus 1.

While the foregoing is directed to exemplary embodiments of the present invention, it will be understood that the present invention is described herein purely by way of example, and modifications of detail can be made within the scope of the invention. Furthermore, one skilled in the art will understand that the present invention may not be limited by the embodiments disclosed herein, or to any details shown in the accompanying figures that are not described in detail herein or defined in the claims.

Moreover, other and further embodiments of the present invention will be apparent to those skilled in the art from consideration of the specification, and may be devised without departing from the basic scope thereof, which is determined by the claims that follow.

1. A nebuliser assembly for a disinfecting apparatus, comprising:
a nebuliser comprising:
an atomisation plate having a front surface and a back surface and a plurality of holes extending therebetween;
a transducer configured to vibrate the atomisation plate;
a fluid retaining substrate arranged against the back surface of the atomisation plate, such that during vibration of the atomisation plate, fluid retained in the fluid retaining substrate is forced through the plurality of holes and ejected from the front surface of the atomisation plate.

2. The nebuliser assembly of claim 1, wherein the fluid retaining substrate comprises a sponge material.

3. The nebuliser assembly of claim 1, wherein the fluid retaining substrate comprises a layer of material.

4. The nebuliser assembly of claim 1, further comprising a moisture sensor arranged to detect moisture in the fluid retaining substrate.

5. The nebuliser assembly of claim 4, wherein the moisture sensor is a binary sensor configured to detect the presence or absence of disinfecting fluid in the fluid retaining substrate.

6. The nebuliser assembly of claim 5, wherein the moisture sensor comprises a first electrode and a second electrode in contact with the fluid retaining substrate, and the moisture sensor is configured to detect electrical current between the electrodes.

7. The nebuliser assembly of claim 1, further comprising an attachment system to enable the nebuliser assembly to be releasably attached to the disinfecting apparatus.

8. The nebuliser assembly of claim 7, wherein the attachment system comprises a magnetic attachment system.

9. The nebuliser assembly of claim 8, wherein the magnetic attachment system comprises a plurality of magnets.

10. The nebuliser assembly of claim 1, further comprising a casing that at least partially encloses the fluid retaining substrate.

11. The nebuliser assembly of claim 10, wherein the casing is configured to at least partially compress the fluid retaining substrate against the atomisation plate.

12. The nebuliser assembly of claim 1, wherein the nebuliser assembly comprises an opening that is aligned with the position of the fluid retaining substrate, such that the fluid can be supplied to the fluid retaining substrate via the opening.

13. The nebuliser assembly of claim 1, wherein the nebuliser assembly comprises one or more additional nebulisers, each comprising a corresponding atomisation plate, and transducer.
